(19) United States
(12) Patent Application Publication
(10) Pub. No.: US 2019/0333399 A1
(43) Pub. Date: Oct. 31, 2019

(54) SYSTEM AND METHOD FOR VIRTUAL REALITY TRAINING USING ULTRASOUND IMAGE DATA

(71) Applicant: General Electric Company, Schenectady, NY (US)

(72) Inventors: Menachem Halmann, Wauwatosa, WI (US); Cynthia Owen, Powhatan, AR (US)

(21) Appl. No.: 15/962,734

(22) Filed: Apr. 25, 2018

(51) Int. Cl. G09B 5/02; G06T 7/00; G06T 11/60; A61B 8/08; A61B 8/00; G09B 23/28

(52) U.S. Cl. CPC G09B 5/02; G06T 7/0012; G06T 11/60; A61B 8/481; A61B 8/08; G06T 2207/20221; A61B 8/5215; A61B 8/463; G09B 23/286; G06T 2207/10132; G06T 2207/30061; A61B 8/5292

(57) ABSTRACT

A medical image training system includes an ultrasound imaging probe configured to acquire ultrasound image data of an imaged body of a person. The system also includes one or more processors configured to obtain one or more imaged pathological structures and to blend the one or more imaged pathological structures with the ultrasound image data to create composite image data. The one or more processors also are configured to direct an output device to display the composite image data to an operator.

### FIELD

The subject matter disclosed herein relates generally to imaging systems.

### BACKGROUND

Imaging systems generate image data representative of imaged bodies. Some imaging systems are not real-time diagnosis or examination modalities in that the image data from these types of systems is obtained, otherwise presented as images or videos at a later time (subsequent to acquisition of the image data), and then presented to an operator for examination.

Other imaging systems are real-time diagnosis or examination modalities in that the image data from these types of systems is obtained and presented for diagnosis or examination by the operator in real-time. For example, the image data of a body can be visually presented to the operator for diagnosis or other examination while the imaging system continues obtaining additional image data of the same body.

One issue with real-time imaging modalities is that operators may miss one or more items of interest in the image data during examination. An operator may manually control a component of the imaging system (e.g., an imaging probe) to acquire the image data while the same operator also is visually inspecting the image data to identify the items of interest, such as regions of the image data that may represent an infection or diseased portion of the imaged body. This can result in the operator missing one or more items of interest in the image data, especially in operators that have less experience or training than other operators.

### BRIEF DESCRIPTION

In one embodiment, a medical image training system includes an ultrasound imaging probe configured to acquire ultrasound image data of an imaged body of a person. The system also includes one or more processors configured to obtain one or more imaged pathological structures and to blend the one or more imaged pathological structures with the ultrasound image data to create composite image data. The one or more processors also are configured to direct an output device to display the composite image data to an operator.

In one embodiment, a method includes acquiring ultrasound image data of an imaged body of a person, obtaining one or more imaged pathological structures associated with one or more of a disease or an infection, blending the one or more imaged pathological structures with the ultrasound image data to create composite image data, and displaying the composite image data to an operator.

In one embodiment, a method includes acquiring ultrasound image data of an imaged body, obtaining one or more previously imaged pathological structures associated with one or more of a disease or an infection, blending the one or more previously imaged pathological structures with the ultrasound image data to create composite image data, displaying the composite image data to an operator, receiving a user identification of the one or more previously imaged pathological structures, and determining whether the user identification includes an accurate medical diagnosis by comparing the user identification with a designated diagnosis identification that is associated with the one or more previously imaged pathological structures that are blended with the ultrasound image data.

### BRIEF DESCRIPTION OF THE DRAWINGS

The inventive subject matter described herein will be better understood from reading the following description of non-limiting embodiments, with reference to the attached drawings, wherein below:

FIG. 1 is a schematic diagram of an ultrasound imaging system in accordance with one embodiment of the inventive subject matter described herein;

FIG. 2 illustrates a thoracic cavity of a person according to one example;

FIG. 3 illustrates one embodiment of an ultrasound probe of the ultrasound imaging system shown in FIG. 1;

FIG. 4 illustrates a flowchart of one embodiment of a method for training an operator to identify pathological structures in ultrasound image data using virtual reality image data;

FIG. 5 illustrates one example of ultrasound image data having a visible pathological structure;

FIG. 6 illustrates one example of unhealthy ultrasound image data;

FIG. 7 illustrates another example of healthy ultrasound image data;

FIG. 8 illustrates blending of an extracted image data portion of unhealthy image data with healthy image data shown in FIG. 8 to form composite image data;

FIG. 9 also illustrates the blending of the extracted image data portion of unhealthy image data with the healthy image data shown in FIG. 8 to form composite image data; and

FIG. 10 illustrates one example of a graphical user interface that can be shown on a display device of the imaging system shown in FIG. 1 to present the composite image data to the operator of the imaging system.

### DETAILED DESCRIPTION

One or more embodiments of the inventive subject matter described herein provide imaging systems and methods that obtain real-time image data of a body and add in virtual reality image data representative of pathological structures to the image data for presentation to an operator of the imaging system. The real-time image data can be image data that is acquired and displayed to the operator while additional image data of the body is acquired. The pathological structures can represent infected, damaged, or diseased areas of a different body. The systems and methods can add the pathological structures obtained from previously imaging a damaged, diseased, or infected body of a first person (e.g., a person that is known to be unhealthy) to the image data acquired for a different, second person (e.g., a person that is known to be healthy) person so that the operator that is imaging the second person can attempt to identify the added pathological structures in the image data of the healthy person. This can help to train the operator to identify pathological structures more accurately and/or more quickly, which will help the operator when subsequently imaging a person that is not known to be healthy or unhealthy. In one embodiment, the image data is ultrasound image data.

At least one technical effect of the inventive subject matter described herein includes the improved training of operators in identifying structures of interest in real-time image data during examination of a person. The systems and methods can be used to improve the ability of the operator to accurately and quickly identify pathological structures in a person using real-time image data, with reduces instances of false-positive diagnoses and/or missed diagnoses.

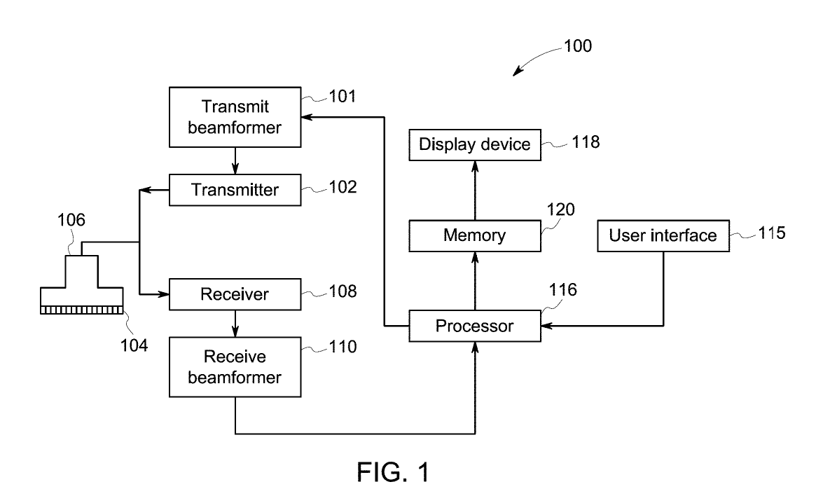

FIG. 1 is a schematic diagram of an ultrasound imaging system 100 in accordance with one embodiment of the inventive subject matter described herein. The ultrasound imaging system 100 includes a transmit beamformer 101 and a transmitter 102 that drive elements 104 within a probe 106 to emit pulsed ultrasonic signals into a body (not shown). According to an embodiment, the probe 106 may be a two-dimensional matrix array probe. Another type of probe capable of acquiring four-dimensional ultrasound data may be used according to one or more other embodiments. The four-dimensional ultrasound data can include ultrasound data such as multiple three-dimensional volumes acquired over a period of time. The four-dimensional ultrasound data can include information showing how a three-dimensional volume changes over time.

The pulsed ultrasonic signals are back-scattered from structures in the body, such as blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. The probe 106 may contain electronic circuitry to do all or part of the transmit and/or the receive beamforming. For example, all or part of the transmit beamformer 101, the transmitter 102, the receiver 108 and the receive beamformer 110 may be situated within the probe 106. Scanning may include acquiring data through the process of transmitting and receiving ultrasonic signals. Data generated by the probe 106 can include one or more datasets acquired with an ultrasound imaging system. A user interface 115 may be used to control operation of the ultrasound imaging system 100, including, to control the input of person data, to change a scanning or display parameter, and the like.

The ultrasound imaging system 100 also includes one or more processors 116 that control the transmit beamformer 101, the transmitter 102, the receiver 108 and the receive beamformer 110. The processors 116 are in electronic communication with the probe 106 via one or more wired and/or wireless connections. The processors 116 may control the probe 106 to acquire data. The processors 116 control which of the elements 104 are active and the shape of a beam emitted from the probe 106. The processors 116 also are in electronic communication with a display device 118, and the processors 116 may process the data into images for display on the display device 118. The processors 116 may include one or more central processors (CPU) according to an embodiment. According to other embodiments, the processors 116 may include one or more other electronic components capable of carrying out processing functions, such as one or more digital signal processors, field-programmable gate arrays (FPGA), graphic boards, and/or integrated circuits. According to other embodiments, the processors 116 may include multiple electronic components capable of carrying out processing functions. For example, the processors 116 may include two or more electronic components selected from a list of electronic components including: one or more central processors, one or more digital signal processors, one or more field-programmable gate arrays, and/or one or more graphic boards. According to another embodiment, the processors 116 may also include a complex demodulator (not shown) that demodulates the radio frequency data and generates raw data. In another embodiment, the demodulation can be carried out earlier in the processing chain.

The processors 116 are adapted to perform one or more processing operations according to a plurality of selectable ultrasound modalities on the data. The data may be processed in real-time during a scanning session as the echo signals are received, such as by processing the data without any intentional delay or processing the data while additional data is being acquired during the same imaging session of the same person. For example, an embodiment may acquire images at a real-time rate of seven to twenty volumes per second. The real-time volume-rate may be dependent on the length of time needed to acquire each volume of data for display, however. Accordingly, when acquiring a relatively large volume of data, the real-time volume-rate may be slower. Some embodiments may have real-time volume-rates that are considerably faster than twenty volumes per second while other embodiments may have real-time volume-rates slower than seven volumes per second.

The data may be stored temporarily in a buffer (not shown) during a scanning session and processed in less than real-time in a live or off-line operation. Some embodiments of the inventive subject matter may include multiple processors (not shown) to handle the processing tasks that are handled by the processors 116 according to the exemplary embodiment described hereinabove. For example, a first processor may be utilized to demodulate and decimate the RF signal while a second processor may be used to further process the data prior to displaying an image. It should be appreciated that other embodiments may use a different arrangement of processors.

The ultrasound imaging system 100 may continuously acquire data at a volume-rate of, for example, ten to thirty hertz. Images generated from the data may be refreshed at a similar frame-rate. Other embodiments may acquire and display data at different rates. For example, some embodiments may acquire data at a volume-rate of less than ten hertz or greater than thirty hertz depending on the size of the volume and the intended application.

A memory 120 is included for storing processed volumes of acquired data. In one embodiment, the memory 120 is of sufficient capacity to store at least several seconds worth of volumes of ultrasound data. The volumes of data are stored in a manner to facilitate retrieval thereof according to its order or time of acquisition. The memory 120 may comprise any known data storage medium, such as one or more tangible and non-transitory computer-readable storage media (e.g., one or more computer hard drives, disk drives, universal serial bus drives, or the like).

Optionally, one or more embodiments of the inventive subject matter described herein may be implemented utilizing contrast agents. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component and generating an ultrasound image by utilizing the enhanced harmonic component. Separation of harmonic components from the received signals is performed using suitable filters.

In various embodiments of the present invention, data may be processed by other or different mode-related modules by the processors 116 (e.g., B-mode, Color Doppler, M-mode, Color M-mode, spectral Doppler, Elastography, TVI, strain, strain rate, and the like) to form two- or three-dimensional image data. For example, one or more modules may generate B-mode, color Doppler, M-mode, color M-mode, spectral Doppler, Elastography, TVI, strain, strain rate and combinations thereof, and the like. The image beams and/or volumes are stored and timing information indicating a time at which the data was acquired in memory may be recorded. The modules may include, for example, a scan conversion module to perform scan conversion operations to convert the image volumes from beam space coordinates to display space coordinates. A video processor module may read the image volumes from a memory and displays an image in real time while a procedure is being carried out on a person. A video processor module may store the images in an image memory, from which the images are read and displayed.

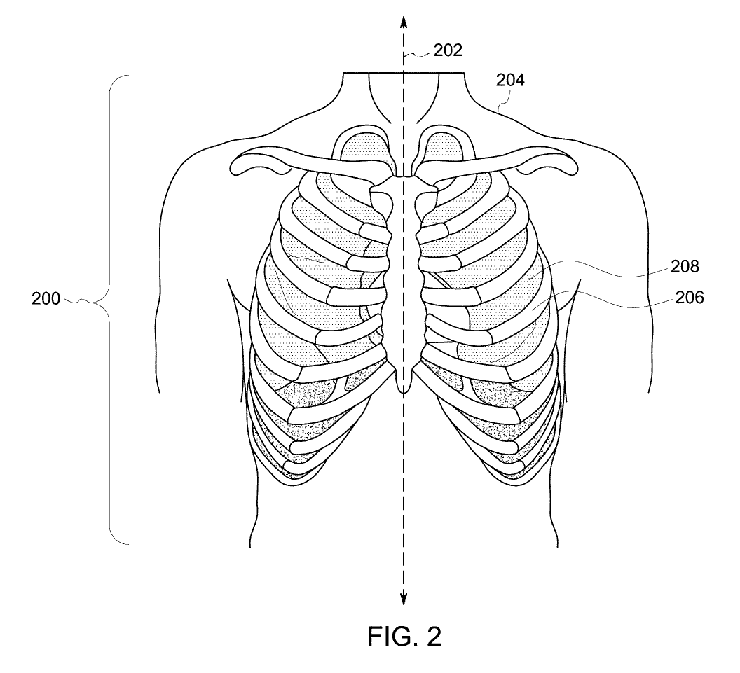

FIG. 2 illustrates a thoracic cavity 200 of a person 204 according to one example. The ultrasound image data that is obtained and used to train operators (as described herein) may represent portions of the thoracic cavity 200, including lungs 208 and one or more ribs 206 of the person 204. In obtaining the ultrasound image data, the probe 106 shown in FIG. 1 may be held in contact with an exterior surface of the skin of the person 204 and moved transversely to the ribs 206. For example, the probe 106 may be moved in a direction that is parallel or substantially parallel to the sagittal plane 202 of the person 204 (e.g., within ten degrees of parallel, within 15 degrees of parallel, etc.). As the probe 106 is moved in this direction during acquisition of ultrasound image data, the probe 106 moves transverse or substantially transverse to directions in which the various ribs 206 are elongated.

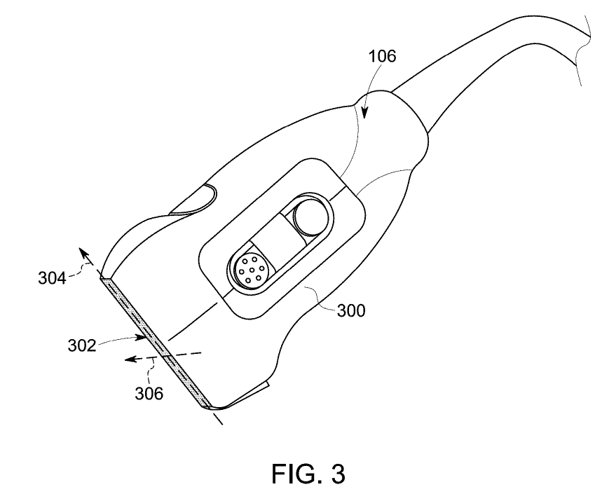

FIG. 3 illustrates one embodiment of the probe 106 of the ultrasound imaging system 100 shown in FIG. 1. The probe 106 can have a housing 300 that holds the drive elements 104 (not visible inside the housing 300 in FIG. 3). The housing 300 of the probe 106 interfaces (e.g., contacts) the person 204 along a face surface 302 of the housing 300. This face surface 302 is elongated along a first direction 304 relative to an orthogonal (e.g., perpendicular) direction 306.

The probe 106 can be moved along the outside of the person 204 along the thoracic cavity 200 to acquire ultrasound image data of the lungs 208 of the person 204. In one embodiment, the probe 106 is moved transversely to directions in which the ribs 206 are elongated. For example, the probe 106 can be moved along the exterior of the person 204 in directions that are more parallel to the sagittal plane 202 than perpendicular to the sagittal plane 202.

The probe 106 can be held in an orientation that has the elongated direction 304 of the housing 300 of the probe 106 oriented parallel to (or more parallel than perpendicular) the ribs 206 of the person 204 while the probe 106 is moved along the sagittal plane 202. This orientation of the probe 106 can be referred to as a sagittal position or orientation of the probe 106. Alternatively, the probe 106 can be held in an orientation that is perpendicular to the sagittal orientation. This orientation results in the probe 106 being oriented such that the elongated direction 304 of the housing 300 of the probe 106 is perpendicular to (or more perpendicular than parallel) the ribs 206 of the person 204 while the probe 106 is moved along the sagittal plane 202. This orientation of the probe 106 can be referred to as a transverse position or orientation of the probe 106.

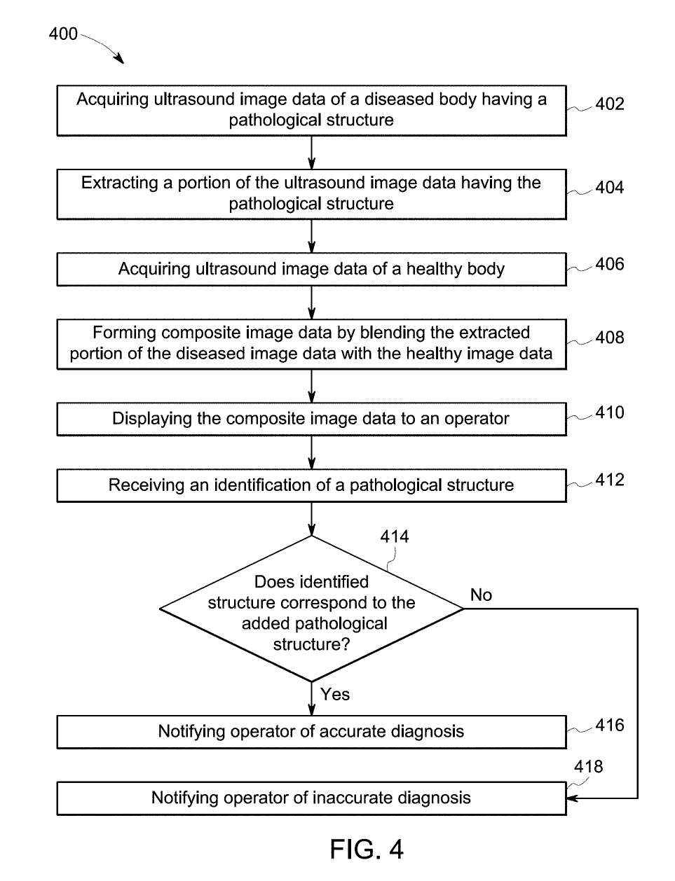

FIG. 4 illustrates a flowchart of one embodiment of a method 400 for training an operator to identify pathological structures in ultrasound image data using virtual reality image data. The method 400 represents operations performed by the ultrasound imaging system 100 to acquire ultrasound image data of a first person having known or identified pathological structures. These pathological structures can be diseased, infected, or otherwise damaged internal structures of the first person. As described below, the system 100 and method 400 can take portions of the image data showing the pathological structures and add these portions of the image data to ultrasound image data acquired from a different, second person. This second person may be a healthy person, a volunteer, or the operator that does not have the same pathological structures as the first person. The pathological structure portions of the image data from the first (unhealthy) person can be blended into the image data of the second (healthy) person to form composite image data. This composite image data can be displayed to an operator of the imaging system 100 in real time (as the image data of the healthy person continues to be obtained and displayed) to help train the operator to better identify pathological structures in the image data.

At 402, ultrasound image data of a diseased body having a pathological structure is acquired. The ultrasound imaging system 100 (or another ultrasound imaging system) can image a person 204 having a known or previously diagnosed infection, disease, or other bodily damage. One or more pathological structures indicative of this infection, disease, or other bodily damage appear in the acquired ultrasound image data. For example, the memory 120 of the system 100 shown in FIG. 1 may store many different sets of ultrasound image data previously acquired from imaging scans of the same person 204 (when the person was unhealthy) and/or other persons. This ultrasound image data may have been previously examined and pathological structures identified in the ultrasound image data. The pathological structures may have been identified and extracted from the ultrasound image data, with the extracted portions of the image data stored in the memory 120.

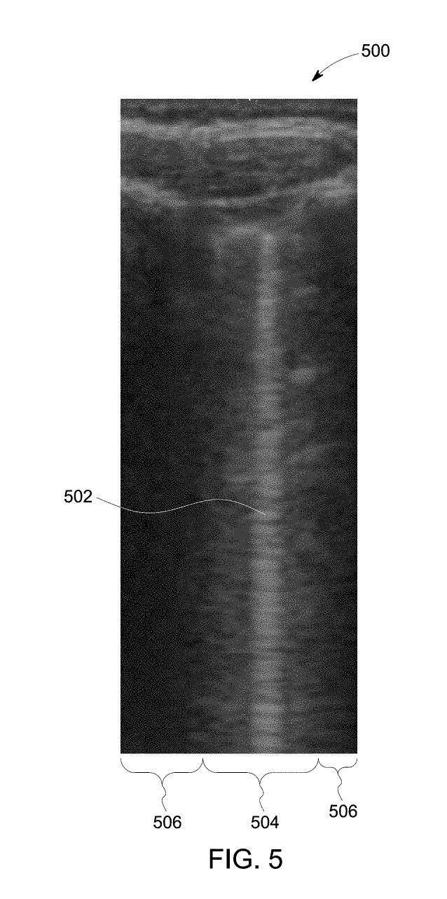

FIG. 5 illustrates one example of ultrasound image data 500 having a visible pathological structure 502. The ultrasound image data 500 shows a portion of an intercostal space 504 between ribs 206 of an unhealthy person. The image data 500 also shows parts of rib shadows 506 on either side of the intercostal space 504. These shadows 506 indicate where passage of the pulsed ultrasonic signals was blocked by the ribs 206.

The pathological structure 502 appears in the ultrasound image data 500 as a B-line, which is a predominantly vertical line in the ultrasound image data 500. This B-line can indicate that the intercostal space 504 of the lung of the person that appears in the image data 500 is infected, such as due to the person suffering from pneumonia.

personperson Returning to the description of the flowchart of the method 400 shown in FIG. 4, the ultrasound image data 500 in FIG. 5 provides some non-limiting examples of the types of ultrasound image data having pathological structures that is acquired at 402. Alternatively, ultrasound image data having other pathological structures, such as tumors, follicles, or the like, may be acquired.

At 404, a portion of the ultrasound image data having the pathological structure can be extracted from the ultrasound image data. In one embodiment, an operator of the imaging system 100 can use the user interface 115 (e.g., a touchscreen, electronic mouse or stylus coupled with a display device, etc.) to select the area or areas in the previously acquired ultrasound image data where the pathological structures appear. With respect to the example shown in FIG. 5, the operator can trace a perimeter around the B-lines representing the pathological structure 502.

Alternatively, the processor 116 of the imaging system 100 can automatically or semi-automatically identify and select the area or areas in the image data in which the pathological structures appear. With respect to automatically identifying the pathological structure, the processor 116 can examine characteristics of pixels in the image data 500 to identify where the pathological structures are located without operator intervention. This can involve the processor 116 identifying a group of interconnected or neighboring pixels having an intensity, color, or other characteristic that is within a designated range of each other, and optionally where the average, median, or mode characteristic of the pixels in the group differs from pixels outside the group of pixels (e.g., by at least a threshold amount). For example, the processor 116 can identify boundaries between groups of pixels having different characteristics, with the group of pixels that is enclosed (e.g., by a closed perimeter of other group or groups of pixels) representing a pathological structure.

With respect to semi-automatically identifying the pathological structure(s), an operator can select the areas in the image data where the pathological structure(s) appear, and the processor 116 can use this operator identification to determine boundaries between groups of pixels having different characteristics, with the group of pixels that is enclosed and selected by the operator representing a pathological structure.

The processor 116 can extract the portion of the image data that shows the identified pathological structure by removing or copying the portion of the image data associated with the pixels (or voxels) included in the identified pathological structure. This portion of the image data having the pathological structure can be stored in the memory 120. Optionally, instead of removing or copying the pathological structure portion of the image data from the image data, the entire image data can be stored (including the portions with and without the pathological structure) with data stored in or with the image data that identifies where the pathological structure is located. For example, metadata can be stored with the image data to identify which pixels or areas in the image data represent the pathological structure.

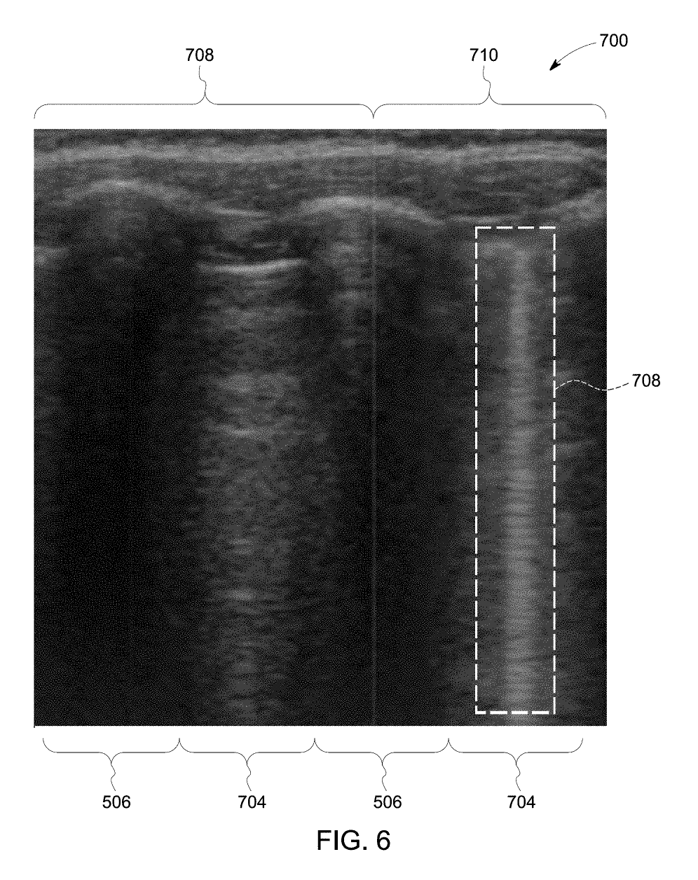

FIG. 6 illustrates one example of unhealthy ultrasound image data 700. The ultrasound image data 700 may be obtained from a diseased person that is suffering from disease, infection, or other damage. The unhealthy image data 700 shows a panoramic view of several intercostal spaces 704 between ribs 206 of the person. The image data 700 also shows rib shadows 506 on either side of the intercostal spaces 704. As shown, a pathological structure 706 appears in the image data 700. This pathological structure 706 appears as a vertical b-line within one of the intercostal spaces 704 that is brighter than the remaining portion of the intercostal space 704. This pathological structure 706 can represent an infection, damage, or disease, such as pneumonia, in the lung. The dashed line box drawn around the pathological structure 706 is provided to aid the reader is seeing the structure 706, and may not appear in the image data 700 that is shown on the display device 118.

The panoramic view shown in FIG. 6 can be acquired by obtaining portions 708, 710 of the image data 700 of different intercostal spaces 704 as the probe 106 is transversely moved over the ribs 206 of the person, and then stitching or otherwise combining these different image data portions 708, 710 together. For example, the image data portion 708 may be acquired before the image data portion 710 as the probe 106 moves over the thoracic cavity 200 of the person 204. The different image data portions 708, 710 can then be combined by the processor 116 to form the view shown in FIG. 6.

Returning to the description of the flowchart of the method 400 shown in FIG. 4, at 406, ultrasound image data of a healthy body is acquired. This image data can be referred to as healthy image data, while the image data having the pathological structure(s) can be referred to as unhealthy or diseased image data.

The same or different imaging system 100 that acquired the image data having the pathological structure(s) can be used to acquire the ultrasound data of the healthy body. The body may be healthy in that the body may not have the same disease, infection, damage, or the like, of the diseased body. Optionally, the body may be healthy in that the body may have some disease, infection, damage, or the like, but not the same disease, infection, damage, or the like, in the same location as the diseased body. The body from which the healthy image data is obtained can be a different body than the body from which the diseased image data was acquired. Alternatively, the body from which the healthy image data is obtained can be the same body from which the diseased image data was acquired. For example, the diseased image data can be obtained from a lung of a person when the person previously was ill, while the healthy image data can be obtained from the same or other lung of the same person after the person has healed from the illness.

The healthy image data can be acquired by an operator of the imaging system 100 that is being trained to increase the aptitude of the operator and/or to test the aptitude of the operator to recognized and identify pathological structures in the ultrasound image data. As described herein, the pathological structures obtained from the diseased image data can be blended into the healthy image data to create the appearance that the healthy image data actually shows the pathological structures. This can allow for the operator to practice identifying a wide variety of diseases, infections, damage, or the like, in image data acquired of healthy persons that do not currently suffer from the disease, infection, damage, or the like.

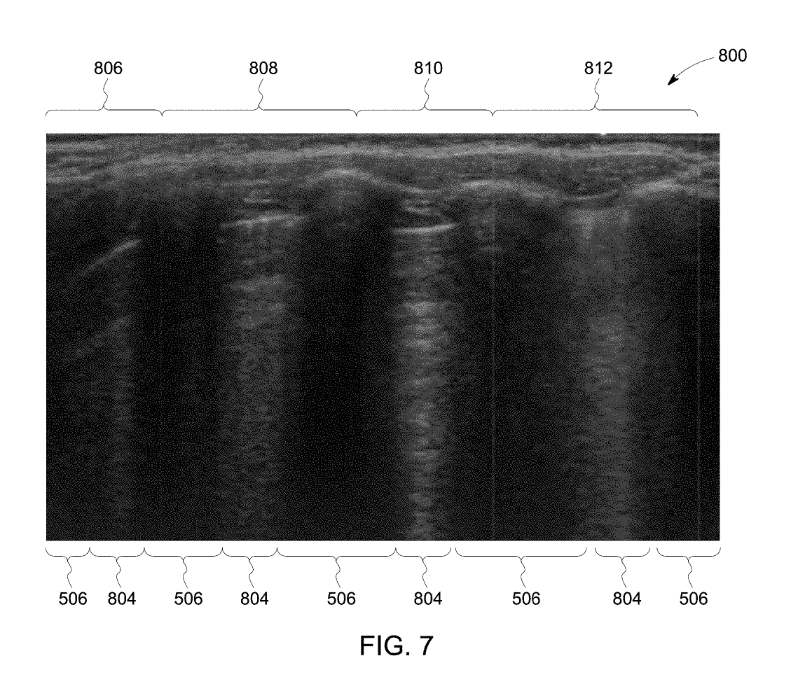

FIG. 7 illustrates another example of healthy ultrasound image data 800. The ultrasound image data 800 may be obtained from a healthy person that is not suffering from the disease, infection, or other damage that the person from which the diseased image data 700 shown in FIG. 6 was obtained. The healthy image data 800 shows a panoramic view of several intercostal spaces 704 between ribs 206 of a healthy person. The image data 800 also shows rib shadows 506 on either side of the intercostal spaces 804. As shown, no pathological structures appear in the healthy image data 800. The panoramic view shown in FIG. 7 can be acquired by obtaining portions 806, 808, 810, 812 of the image data 800 of different intercostal spaces 804 as the probe 106 is transversely moved over the ribs 206 of the person, and then stitching or otherwise combining these different image data portions 806, 808, 810, 812 together.

For example, the image data portion 806 may be acquired before the image data portions 808, 810, 812 as the probe 106 moves over the thoracic cavity 200 of the person 204, the image data portion 808 may be acquired before the image data portions 810, 812 as the probe 106 moves over the thoracic cavity 200 of the person 204, and the image data portion 810 may be acquired before the image data portion 812 as the probe 106 moves over the thoracic cavity 200 of the person 204. The different image data portions 806, 808, 810, 812 can then be combined by the processor 116 to form the view shown in FIG. 7.

Returning to the description of the flowchart of the method 400 shown in FIG. 4, at 408, composite image data is formed by blending the extracted portion of the diseased image data with the healthy image data. The extracted portion of the diseased image data includes the part of the diseased image data representing the pathological structure(s). The processor 116 can blend the extracted portion of the diseased image data to the healthy image data by adding the extracted image portion to the healthy image data and/or replacing a portion of the healthy image data with the extracted image portion. This causes the pathological structure(s) in the extracted image portion to virtually appear in the healthy image data.

The extracted portion of the diseased image data can be blended into the healthy image data in real time. For example, the extracted portion can be added to the healthy image data before the imaging system 100 displays or otherwise presents the combination of the extracted portion and the healthy image data. This can result in the composite ultrasound image data that is presented to the operator appear to be ultrasound image data representative of pathological structures in the imaged body that are not in the imaged body.

The processor 116 can add the extracted image data containing the pathological structures with the healthy image data so that the pathological structures appear to be inside the healthy body being imaged. The processor 116 optionally can change one or more characteristics of the extracted image data so that the pathological structures more closely match the characteristics of the healthy image data.

As one example, the brightness or intensity of the extracted image data can be different from the healthy image data. This can cause the pathological structures to appear brighter (or darker) than the surrounding portions of the healthy image data. The processor 116 can examine the brightness or intensity of the pixels or voxels in the extracted image data and can examine the brightness or intensity of the pixels or voxels in the extracted image data. If the extracted image brightness or intensity is within a designated range or amount of the healthy image brightness or intensity, then the processor 116 may not need to change the brightness or intensity of the extracted image data. For example, if the average, median, and/or mode of the brightness or intensity of the extracted image data is within 1% (or within 3%, within 5%, within 10%, or within another user-definable range or limit) of the average, median, and/or mode of the brightness or intensity of the healthy image data, then the processor 116 may not change the brightness or intensity of the extracted image data (and/or the healthy image data).

But, if the extracted image brightness or intensity is not within this designated range of the healthy image brightness or intensity, then the processor 116 may change the brightness or intensity of the extracted image data (or, alternatively, of the healthy image data). The processor 116 can decrease (or increase) the brightness or intensity of some or all pixels or voxels in the extracted image data to match or more closely match the brightness or intensity of some or all pixels or voxels in the healthy image data. For example, the processor 116 can decrease the brightness or intensity of the pixels in the extracted image data so that the average, median, or mode of the brightness or intensity of the pixels in the extracted image data is the same as or is within the range of the average, median, or mode of the brightness or intensity of the pixels in the healthy image data (or at least the pixels in the healthy image data that neighbor or are adjacent to the extracted image portion).

As another example, the color of the extracted image data can be different from the healthy image data. This can cause the pathological structures to appear significantly different than the surrounding portions of the healthy image data. The processor 116 can examine the color(s) of the pixels or voxels in the extracted image data and can examine the color(s) of the pixels or voxels in the extracted image data. If values of the extracted image color are within a designated range or amount of the values of the healthy image color (e.g., the tristimulus values, the irradiance values, the reflectance, the transmittance, and/or the color temperatures of the colors), then the processor 116 may not need to change the color(s) of the extracted image data.

But, if the values of the extracted image color are not within this designated range of the values of the healthy image color(s), then the processor 116 may change the color(s) of the extracted image data (or, alternatively, of the healthy image data). The processor 116 can change the values of the colors of some or all pixels or voxels in the extracted image data to match or more closely match the values of the colors of some or all pixels or voxels in the healthy image data. For example, the processor 116 can change the colors of the pixels in the extracted image data so that the average, median, or mode of the values of the colors of the pixels in the extracted image data is the same as or is within the range of the average, median, or mode of the values of the colors of the pixels in the healthy image data (or at least the pixels in the healthy image data that neighbor or are adjacent to the extracted image portion).

The processor 116 can add the extracted image data to the healthy image data so that the extracted image data becomes part of the healthy image data. This can allow for the composite image data to move on the display device 118 with the healthy image data portion of the composite image data and the extracted image data portion of the composite image data moving together (e.g., the same speeds, distances, etc., on the display device 118).

Blending the extracted image data into the healthy image data in one or more of these ways can result in the pathological structures of the extracted image data appearing more natural or as real pathological structures captured in the healthy image data. This can help to better train the operator in recognizing pathological structures in ultrasound image data obtained from other persons at a later time.

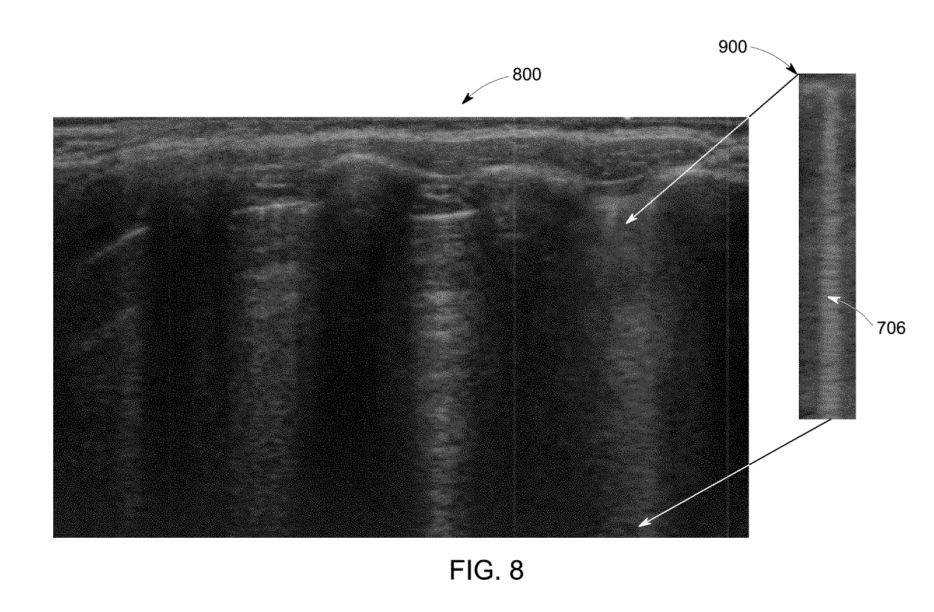

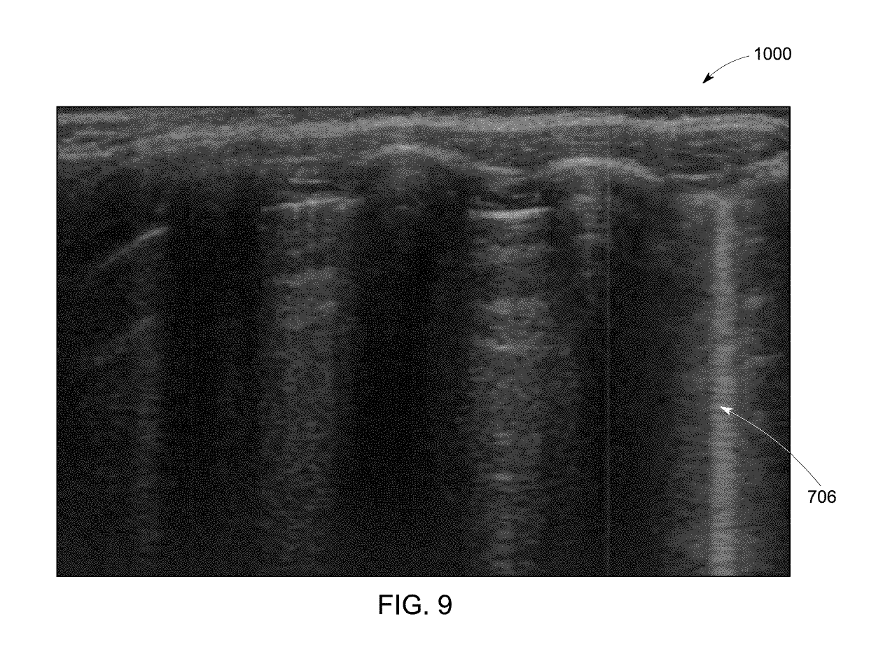

FIGS. 8 and 9 illustrate the blending of an extracted image data portion 900 of unhealthy image data with the healthy image data 800 shown in FIG. 7 to form composite image data 1000. The healthy image data 800 and the composite image data 1000 show panoramic views of several intercostal spaces 704 between ribs 206 of a healthy person. The extracted portion 900 can be added to the healthy image data 800 to form the composite image data 1000.

As shown in FIG. 9, the pathological structure 706 shown in the extracted image data portion 900 appears in the composite image data 1000 as part of the image data 1000. That is, the pathological structure 706 does not appear to have been added by the processor 116 to the healthy image data 800, but appears to have been obtained by the probe 106 as the operator being trained moves the probe 106 to obtain the image data 800.

In one embodiment, the pathological structures that are selected for adding to healthy image data to form the composite image data can be selected based on a training achievement of the operator. Different operators may have different levels of experience due to different lengths of time that the operators have been working on examining ultrasound image data, due to the operators completing different training exercises or classes, or the like. The different experience levels of the operators can be referred to as different training achievements, and can be associated with the operators in the memory 120. For example, the processor 116 can determine the training achievement of an operator when the operator logs into or otherwise accesses the imaging system 100. The training achievement can be stored in the memory 120 in such a way that the training achievement is associated with the operator.

The processor 116 can then select the pathological structure from among many different pathological structures based on the training achievement of the operator. The pathological structures can be selected from a larger set of available pathological structures in the memory 120. For example, the processor 116 can select pathological structures that are larger for operators having less experience (and, therefore, a lower training achievement) and can select smaller pathological structures for operators having more experience (and, therefore, a higher training achievement). As another example, the processor 116 can select a pathological structure that the operator has not yet been trained to identify. As another example, the processor 116 can select a pathological structure that the operator has previously been unable to identify or has not had at least a threshold level of success in identifying (e.g., the operator has incorrectly identified or failed to identify the pathological structure more than a designated number of times or more than a designated percentage of attempts).

Optionally, the operator can select a training difficulty level that dictates which pathological structures are selected by the processor 116 for blending into the healthy image data. The processor 116 can select smaller pathological structures and/or pathological structures that are visible for shorter periods of time for more difficult training levels, and can select larger pathological structures and/or pathological structures that are visible for longer periods of time for less difficult training levels. The pathological structures can be selected from a larger set of available pathological structures in the memory 120. The processor 116 can then blend the selected pathological structures into the healthy image data to form the composite image data, as described herein.

Optionally, the processor 116 can set or change a brightness of the pathological structure that is blended into the healthy image data based on a training level or difficulty level of the operator or selected by the operator. The processor 116 can increase the difference between the pathological structure and the healthy image data for lower training or difficulty levels. For example, the processor 116 can make the pathological structure much brighter than the healthy image data into which the pathological structure is blended to make the pathological easier to identify. Conversely, the processor 116 can decrease the difference between the pathological structure and the healthy image data for higher training or difficulty levels. For example, the processor 116 can make the pathological structure have a brightness that is closer to the healthy image data into which the pathological structure is blended to make the pathological more difficult to identify.

The processor 116 can change how the pathological structures are blended into the healthy image data for different training levels, for different training sessions, and/or for different operators. For example, the processor 116 can change a size and/or orientation of a pathological structure when the pathological structure is blended into healthy image data for different examinations by the same or different operators. This can help prevent the operator or operators from being able to correctly identify the pathological structure due to the operator or operators previously examining the same pathological structure blended into the same healthy image data. As another example, the processor 116 can change a location where the pathological structure is blended into the healthy image data for different training levels, for different training sessions, and/or for different operators.

In another example, the processor 116 can change an injection timing in which the pathological structure appears in the composite image data for different training levels, for different training sessions, and/or for different operators. The injection timing dictates when the pathological structure first appears in the composite image data and/or how long the pathological structure is visible in the composite image data. The processor 116 can change the injection timing for different training levels, for different training sessions, and/or for different operators.

In another example, the processor 116 can change a temporal duration in which the pathological structure appears in the composite image data for different training levels, for different training sessions, and/or for different operators. The temporal duration dictates how long the pathological structure is visible in the composite image data.

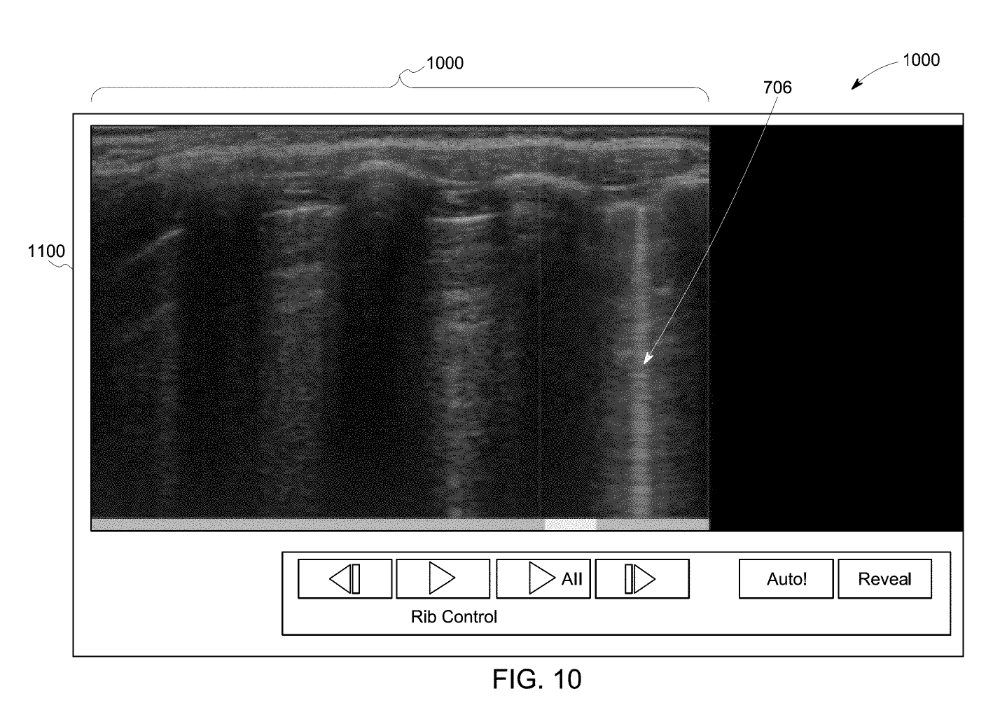

Returning to the description of the flowchart of the method 400 shown in FIG. 4, at 410, the composite image data is displayed to an operator of the imaging system. For example, the composite image data 1000 can be presented to the operator being trained, such as by displaying the composite image data 1000 on the display device 118. The composite image data 1000 can be displayed as the healthy image data 800 continues be obtained by the operator using the probe 106. FIG. 10 illustrates one example of a graphical user interface 1100 that can be shown on the display device 118 to present the composite image data 1000 to the operator of the imaging system 100. The processor 116 can generate and communicate signals to the display device 118 that direct the display device 118 to visually present the composite image data 1000. The user interface 1100 can include one or more buttons or other graphical objects that can be selected by the user (such as the “Reveal” button shown in FIG. 10) to cause the processor 116 to direct the display device 118 to highlight or otherwise point out where the pathological structure is located in the composite image data 1000.

In one embodiment, the composite image data 1000 changes with respect to time such that the pathological structure 706 that was blended into the healthy image data 800 is only shown for a period of time. For example, because ultrasound image data is acquired and shown in real time, the ultrasound image data can be shown on the display device 118 as a video or cine that changes with respect to time. The pathological structure(s) that is or are added to the healthy image data may only appear for some time, but not the entire time, that the composite image data is displayed. The processor 116 may form the composite image data 1000 such that the added pathological structure is only visible while the operator is acquiring image data of a location in the healthy person where the pathological structure is to appear. The pathological structure may no longer appear after the operator moves the probe 106 so that the location where the pathological structure was added is no longer being imaged.

At 412 in the flowchart of the method 400 shown in FIG. 4, an identification of a pathological structure optionally can be received by the imaging system. The operator being trained with the composite image data can use an input device (e.g., a touchscreen, electronic mouse, stylus, keyboard, or the like) to select or otherwise identify an area or areas on the composite image data when the operator believes that he or she sees a pathological structure in the composite image data. The processor 116 of the imaging system 100 can receive this identification as one or more locations in the composite image data 1000. The processor 116 can compare these identified locations with the known locations of the pathological structure 706 that was blended into the healthy image data 800 to form the composite image data 1000. This comparison can allow the processor 116 to determine whether the operator correctly identified the pathological structure 706 that was added to the healthy image data 800.

At 414, a determination is made as to whether the operator-identified structure corresponds with the added pathological structure. The processor 116 can compare the locations on the composite image data that are selected by the operator with the location(s) in which the extracted image data showing the pathological structure was or were added to the healthy image data. If the location(s) identified by the operator is or are the same as the location(s) where the pathological structure was or were blended into the healthy image data, then the operator may have correctly identified the pathological structure in the composite image data. As a result, flow of the method 400 can proceed toward 416. As another example, if the location(s) identified by the operator is or are included in the locations where the pathological structure(s) was or were blended into the healthy image data, then the operator may have correctly identified the pathological structure in the composite image data. As a result, flow of the method 400 can proceed toward 416.

But, if the location(s) identified by the operator are not the same as the location(s) where the pathological structure was or were blended into the healthy image data, then the operator may not have correctly identified the pathological structure in the composite image data. As a result, flow of the method 400 can proceed toward 418. As another example, if the location(s) identified by the operator are not included in the locations where the pathological structure(s) was or were blended into the healthy image data, then the operator may not have correctly identified the pathological structure in the composite image data. As a result, flow of the method 400 can proceed toward 418.

Optionally, the method 400 can include (at 414) determining an incorrect diagnosis responsive to the operator failing to identify the location(s) of the pathological structure(s). The blended pathological structure may only be visible in the composite image data for a short period of time. The processor 116 may not receive any operator identified location(s) at 412, and the processor 116 can determine that the failure of the operator to select one or more locations associated with the pathological structure while the pathological structure is visible on the display device 118 is an incorrect or missed diagnosis. As a result, flow of the method 400 can proceed toward 418.

At 416, the operator is notified of the correct diagnosis. The processor 116 can direct the display device 118 to notify the operator that the operator correctly identified the location of the pathological structure in the composite image data. This notification can be presented responsive to the operator identifying the correct location(s), and can include text, graphics, or the like, shown on the display device 118. Flow of the method 400 can then return toward 402 or optionally may terminate.

At 418, the operator is notified of the incorrect diagnosis. The processor 116 can direct the display device 118 to notify the operator that the operator incorrectly identified the location of the pathological structure in the composite image data. This notification can be presented responsive to the operator identifying the incorrect location(s), and can include text, graphics, or the like, shown on the display device 118. Flow of the method 400 can then return toward 402 or optionally may terminate.

In one embodiment, the pathological structures that are added to healthy image data to form the composite image data can be selected based on a training achievement of the operator. Different operators may have different levels of experience due to different lengths of time that the operators have been working on examining ultrasound image data, due to the operators completing different training exercises or classes, or the like. The different experience levels of the operators can be referred to as different training achievements, and can be associated with the operators in the memory 120. For example, the processor 116 can determine the training achievement of an operator when the operator logs into or otherwise accesses the imaging system 100. The training achievement can be stored in the memory 120 in such a way that the training achievement is associated with the operator.

The processor 116 can then select the pathological structure from among many different pathological structures based on the training achievement of the operator. The pathological structures can be selected from a larger set of available pathological structures in the memory 120. For example, the processor 116 can select pathological structures that are larger for operators having less experience (and, therefore, a lower training achievement) and can select smaller pathological structures for operators having more experience (and, therefore, a higher training achievement). As another example, the processor 116 can select a pathological structure that the operator has not yet been trained to identify. As another example, the processor 116 can select a pathological structure that the operator has previously been unable to identify or has not had at least a threshold level of success in identifying (e.g., the operator has incorrectly identified or failed to identify the pathological structure more than a designated number of times or more than a designated percentage of attempts).

Optionally, the operator can select a training difficulty level that dictates which pathological structures are selected by the processor 116 for blending into the healthy image data. The processor 116 can select smaller pathological structures and/or pathological structures that are visible for shorter periods of time for more difficult training levels, and can select larger pathological structures and/or pathological structures that are visible for longer periods of time for less difficult training levels. The pathological structures can be selected from a larger set of available pathological structures in the memory 120. The processor 116 can then blend the selected pathological structures into the healthy image data to form the composite image data, as described herein.

Optionally, the processor 116 can set or change a brightness of the pathological structure that is blended into the healthy image data based on a training level or difficulty level of the operator or selected by the operator. The processor 116 can increase the difference between the pathological structure and the healthy image data for lower training or difficulty levels. For example, the processor 116 can make the pathological structure much brighter than the healthy image data into which the pathological structure is blended to make the pathological easier to identify. Conversely, the processor 116 can decrease the difference between the pathological structure and the healthy image data for higher training or difficulty levels. For example, the processor 116 can make the pathological structure have a brightness that is closer to the healthy image data into which the pathological structure is blended to make the pathological more difficult to identify.

In one embodiment, a method includes acquiring ultrasound image data of an imaged body of a person, obtaining one or more imaged pathological structures associated with one or more of a disease or an infection, blending the one or more imaged pathological structures with the ultrasound image data to create composite image data, and displaying the composite image data to an operator.

Optionally, the method also includes receiving a user identification of the one or more imaged pathological structures, and determining whether the user identification includes an accurate medical diagnosis by comparing the user identification with a designated diagnosis identification that is associated with the one or more imaged pathological structures that are blended with the ultrasound image data.

Optionally, the one or more imaged pathological structures are blended with the ultrasound image data in real time.

Optionally, the one or more imaged pathological structures are obtained from previous ultrasound imaging of another person.

Optionally, the method also includes selecting the one or more imaged pathological structures that are blended with the ultrasound image data from a larger set of imaged pathological structures based on a training achievement level of the operator.

Optionally, the method also includes selecting the one or more imaged pathological structures that are blended with the ultrasound image data from a larger set of imaged pathological structures based on a training difficulty level selected for the operator.

Optionally, the imaged body includes at least a portion of the person that is not suffering from the one or more of the disease or the infection associated with the one or more imaged pathological structures that are blended with the ultrasound image data.

Optionally, the method also includes notifying the operator of an identity of one or more of the pathological structures, the disease, or the infection.

Optionally, a temporal duration in which the one or more pathological structures are shown in the composite image data is based on a training achievement level of the operator.

Optionally, a brightness level in which the one or more pathological structures are displayed in the composite image data is based on a training achievement level of the operator.

Optionally, the operator is a first operator of two or more operators that view the composite image data, and the method also includes changing one or more of a size or orientation of the one or more pathological structures with respect to time in the composite image data so that the first operator and at least one other operator of the two or more operators view different versions of the composite image data.

Optionally, the operator is a first operator of two or more operators that view the composite image data, and the method also includes changing one or more of a location or an injection timing in which the one or more pathological structures first appear in the composite image data so that the first operator and at least one other operator of the two or more operators view different versions of the composite image data.

In one embodiment, a medical image training system includes an ultrasound imaging probe configured to acquire ultrasound image data of an imaged body of a person. The system also includes one or more processors configured to obtain one or more imaged pathological structures and to blend the one or more imaged pathological structures with the ultrasound image data to create composite image data. The one or more processors also are configured to direct an output device to display the composite image data to an operator.

Optionally, the one or more processors are configured to receive a user identification of the one or more imaged pathological structures via an input device. The one or more processors also can be configured to determine whether the user identification includes an accurate medical diagnosis by comparing the user identification with a designated diagnosis identification that is associated with the one or more imaged pathological structures that are blended with the ultrasound image data.

Optionally, the one or more processors are configured to blend the one or more imaged pathological structures with the ultrasound image data in real time.

Optionally, the one or more processors are configured to select the one or more imaged pathological structures that are blended with the ultrasound image data from a larger set of imaged pathological structures based on a training achievement level of the operator.

In one embodiment, a method includes acquiring ultrasound image data of an imaged body, obtaining one or more previously imaged pathological structures associated with one or more of a disease or an infection, blending the one or more previously imaged pathological structures with the ultrasound image data to create composite image data, displaying the composite image data to an operator, receiving a user identification of the one or more previously imaged pathological structures, and determining whether the user identification includes an accurate medical diagnosis by comparing the user identification with a designated diagnosis identification that is associated with the one or more previously imaged pathological structures that are blended with the ultrasound image data.

Optionally, the one or more previously imaged pathological structures are blended with the ultrasound image data while the ultrasound image data is acquired and displayed to the operator.

Optionally, the method also includes selecting the one or more previously imaged pathological structures that are blended with the ultrasound image data from a larger set of previously imaged pathological structures based on one or more of a training achievement level of the operator or a training difficulty level selected for the operator.

Optionally, the imaged body includes at least a portion of a person not suffering from the one or more of the disease or the infection.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements that do not have that property.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method comprising:
acquiring ultrasound image data of an imaged body of a person;
obtaining one or more imaged pathological structures associated with one or more of a disease or an infection;
blending the one or more imaged pathological structures with the ultrasound image data to create composite image data; and
displaying the composite image data to an operator.

2. The method of claim 1, further comprising:
receiving a user identification of the one or more imaged pathological structures; and
determining whether the user identification includes an accurate medical diagnosis by comparing the user identification with a designated diagnosis identification that is associated with the one or more imaged pathological structures that are blended with the ultrasound image data.

3. The method of claim 1, wherein the one or more imaged pathological structures are blended with the ultrasound image data in real time.

4. The method of claim 1, wherein the one or more imaged pathological structures are obtained from previous ultrasound imaging of another person.

5. The method of claim 1, further comprising selecting the one or more imaged pathological structures that are blended with the ultrasound image data from a larger set of imaged pathological structures based on a training achievement level of the operator.

6. The method of claim 1, further comprising selecting the one or more imaged pathological structures that are blended with the ultrasound image data from a larger set of imaged pathological structures based on a training difficulty level selected for the operator.

7. The method of claim 1, wherein the imaged body includes at least a portion of the person that is not suffering from the one or more of the disease or the infection associated with the one or more imaged pathological structures that are blended with the ultrasound image data.

8. The method of claim 7, further comprising notifying the operator of an identity of one or more of the pathological structures, the disease, or the infection.

9. The method of claim 1, wherein a temporal duration in which the one or more pathological structures are shown in the composite image data is based on a training achievement level of the operator.

10. The method of claim 1, wherein a brightness level in which the one or more pathological structures are displayed in the composite image data is based on a training achievement level of the operator.

11. The method of claim 1, wherein the operator is a first operator of two or more operators that view the composite image data, and further comprising:
changing one or more of a size or orientation of the one or more pathological structures with respect to time in the composite image data so that the first operator and at least one other operator of the two or more operators view different versions of the composite image data.

12. The method of claim 1, wherein the operator is a first operator of two or more operators that view the composite image data, and further comprising:
changing one or more of a location or an injection timing in which the one or more pathological structures first appear in the composite image data so that the first operator and at least one other operator of the two or more operators view different versions of the composite image data.

13. The method of claim 1, wherein the imaged body of the person is a lung and the one or more imaged pathological structures include pneumonia infections.

14. A medical image training system comprising:
an ultrasound imaging probe configured to acquire ultrasound image data of an imaged body of a person; and
one or more processors configured to obtain one or more imaged pathological structures and to blend the one or more imaged pathological structures with the ultrasound image data to create composite image data,
wherein the one or more processors also are configured to direct an output device to display the composite image data to an operator.

15. The system of claim 14, wherein the one or more processors are configured to receive a user identification of the one or more imaged pathological structures via an input device,
wherein the one or more processors also configured to determine whether the user identification includes an accurate medical diagnosis by comparing the user identification with a designated diagnosis identification that is associated with the one or more imaged pathological structures that are blended with the ultrasound image data.

16. The system of claim 14, wherein the one or more processors are configured to blend the one or more imaged pathological structures with the ultrasound image data in real time.

17. The system of claim 14, wherein the one or more processors are configured to select the one or more imaged pathological structures that are blended with the ultrasound image data from a larger set of imaged pathological structures based on a training achievement level of the operator.

18. A method comprising:
acquiring ultrasound image data of an imaged body;
obtaining one or more previously imaged pathological structures associated with one or more of a disease or an infection;
blending the one or more previously imaged pathological structures with the ultrasound image data to create composite image data;
displaying the composite image data to an operator;
receiving a user identification of the one or more previously imaged pathological structures; and
determining whether the user identification includes an accurate medical diagnosis by comparing the user identification with a designated diagnosis identification that is associated with the one or more previously imaged pathological structures that are blended with the ultrasound image data.

19. The method of claim 18, wherein the one or more previously imaged pathological structures are blended with the ultrasound image data while the ultrasound image data is acquired and displayed to the operator.

20. The method of claim 18, further comprising selecting the one or more previously imaged pathological structures that are blended with the ultrasound image data from a larger set of previously imaged pathological structures based on one or more of a training achievement level of the operator or a training difficulty level selected for the operator.

21. The method of claim 18, wherein the imaged body includes at least a portion of a person not suffering from the one or more of the disease or the infection.
